(19) United States
(12) Patent Application Publication
(10) Pub. No.: US 2009/0285885 A1
(43) Pub. Date: Nov. 19, 2009

(54) Method of forming a drug nanocarrier having a magnetic shell

(75) Inventors: San-Yuan Chen, Hsinchu (TW); Shang-Hsiu Hu, Taipei (TW); Dean-Mo Liu, Jhubei (TW)

Correspondence Address: BACON & THOMAS, PLLC, 625 SLATERS LANE, FOURTH FLOOR, ALEXANDRIA, VA, 22314-1176, US

(73) Assignee: National Chiao Tung University, Hsinchu (TW)

(21) Appl. No.: 12/230,973

(22) Filed: Sep. 9, 2008

(30) Foreign Application Priority Data
May 13, 2008 (TW) 097117507

(51) Int. Cl. A61K 9/51; A61K 47/32; A61K 47/02

(52) U.S. Cl. 424/451; 514/772.5; 514/769

(57) ABSTRACT

The invention discloses the synthesis and manufacturing of a novel core-shell nano-carrier with a drug-containing nanocomposite core surrounding with a single crystalline magnetic iron oxide shell. With a unique core-shell configuration, active agents such as drugs and biomolecules encapsulated in the core with an outer single-crystalline thin iron oxide shell can be perfectly protected from environmental damages and in the meantime, eliminating un-desirable release due to un-controllable diffusion of the active molecules from the nanocapsules during the course of delivery in patient's body, before reaching the disease sites.

### BACKGROUND OF THE INVENTION

1. Field of the Invention

This invention is related to a drug nanocarrier, having a core-shell structure, comprising a drug-containing core surrounding with a magnetic-sensitive shell, wherein the said shell is single crystalline, poly-crystalline, or amorphous. Drug can be precisely controlled release while the said nanocarrier is subjecting to a magnetic field.

2. Description of the Prior Art

Controlled release of therapeutic agents or drugs has received increasingly attention in current development of the biomedical industry, especially in the area of developing novel drug delivery technologies. For traditional drug release technology, the drug is generally released under uncontrollable or poorly-designed pattern after administration. The expected therapeutic efficacy is frequently far from perfect and in other words, increase cost of therapeutic practice and burdens of both patients and hospitals. Therefore, it is expected to develop a environmental-stimulating drug-carrier that is able to be used in varying diseases, in order to improved therapeutic efficacy, patient's compliance, and cost.

In the prior art, metal or metal oxide nanoparticles and core-shell configuration has been developed, but the traditional core-shell configuration is often composed of different nano particles. There are channels among nano particles, and thus, the drug is unable to be encapsulated perfectly. In addition, there is natural diffusion phenomenon for the conventional drug container under unchanged external environment. This situation is not ideal for the drug system required to be implanted in the human body for a long time. Thus it is still necessary to develop a completely different drug-carrier system from the traditional technology, which can reach the demand of “Zero-Release” under non-stimulus state. Thus, in order to respond the demand of drug release technology, it is necessary to develop relevant nanocapsule technology to control drug release. At present, no nanocapsule with single-crystal shell was developed and fabricated to form the core-shell nanocapsules by using metal oxide with polymer-directed. With the nano single-crystal shell configuration, the drug release can be effectively controlled and high drug encapsulating efficiency can be made by modifying the dimension of the nanocapsules The development can save the cost such as manpower and time etc.

### SUMMARY OF THE INVENTION

In accordance with the present invention, an apparatus is provided for drug container.

The foregoing, as well as additional objects, features and advantages of the invention will be more readily apparent from the following detailed description, which proceeds with reference to the accompanying drawings.

The present invention relates to a novel core-shell nano-carrier having a drug-containing nanocomposite core surrounded with a single-crystal magnetic iron oxide shell. The nano structure of the present invention is targeted by using the polymer to induce the crystal growing on the core to form perfect single-crystal shell.

The organic material/inorganic material and drug molecules are first reacted to form a drug-containing nanocomposite core structure. Then, the precursor ions of the reactant are grown on the core surface of nanocomposite via polymer targeting by controlling the concentration, time and temperature of reactant to form a nano drug-carrier capsule having the magnetic single-crystal shell.

The present invention not only can encapsulate a great amount of drugs, but also can utilize the unique nano single-crystal structure to encapsulate drugs or biomolecules into a single crystalline magnetic iron oxide shell, so that the carried drugs can reach the goal of zero-release completely.

The drug-carrier capsule with the magnetic nano single-crystal shell has an excellent magnetic sensitivity. A great amount of drug can be released quickly and precisely by the control of magnetic field. When the magnetic field is not, the drug can be encapsulated in the core by the carrier continuously, and the release speed of drug and dose of drug can be controlled, which has excellent advantage for the control of drug release in long or short time.

The drug-carrier of the present invention can be made at room temperature, which will not destroy the activity of drug. The drug-carrier capsule with the magnetic nano single-crystal shell has well-aligned crystal lattice and even thickness.

The drug-carrier capsule with the magnetic nano single-crystal shell has an excellent magnetic sensitivity. A great amount of drug can be released quickly and precisely by the control of magnetic field. When the magnetic field is not applied to the drug-carrier, the drug can be encapsulated in the core by the carrier continuously. This feature is excellent for the long-term drug control, which can be applied in the fields of cancel therapy and drug delivery etc.

The drug-carrier capsule with the magnetic nano single-crystal shell can be used in drug delivery system, and it is better than the drug delivery system developed currently. Therefore, the advantage and spirit of the present invention can be understood further through the following description and attached figures.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows the flowchart diagram of the preferred embodiment for present invention.

FIG. 2 shows the schematic flow diagram of the present invention.

FIGS. 3(a), 3(b) show the transmission electron microscopy image of the present invention.

FIG. 4 shows the sensitive feature of the drug-carrier before and after magnetic stimuli of the present invention.

FIG. 5 shows the fast drug-release behavior of the present invention.

FIG. 6 shows the negligibly small released amount from the nano drug-carrier in the absence of the stimulus to demonstrate the zero-releasing of drug. and,

FIG. 7 shows drug release curves for different size nanoparticles under magnetic stimuli.

### DESCRIPTION OF THE PREFERRED EMBODIMENT

The following is a description of the present invention. The invention firstly will be described with reference to one exemplary structure. Some variations will then be described as well as advantages of the present invention. A preferred method of fabrication will then be discussed. An alternate, asymmetric embodiment will then be described along with the variations in the process flow to fabricate this embodiment.

The present invention relates to a novel core-shell nano-carrier having a drug-containing nanocomposite core surrounding with a single-crystal magnetic iron oxide shell. The preferred embodiments of the present invention are described as follows:

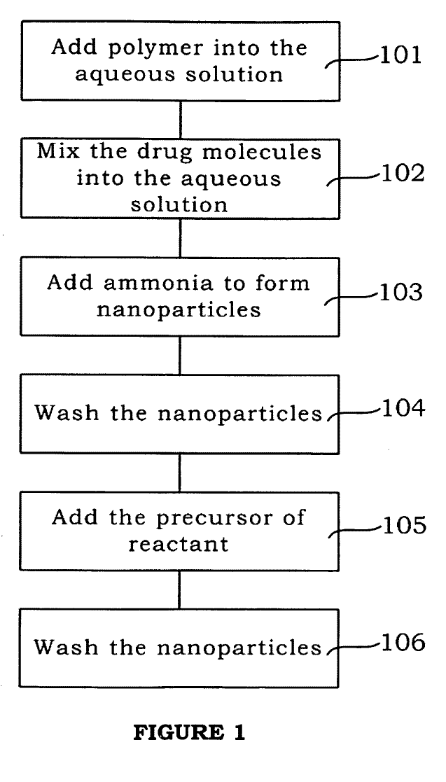

The first embodiment of the present invention is shown in Step 101 of FIG. 1. Firstly, the polymer is added, such as the Polyvinylpyrrolidone (PVP) and Tetraethoxy orthosilane (TEOS) is dissolved in water.

As shown in Step 102 of FIG. 1, the drug molecules (the fluorescence molecules can be simulated as the drug molecules) are mixed with the aforesaid aqueous solution to conduct the hydrolysis for several hours.

As shown in Step 103 of FIG. 1, the ammonia is added to form silicon dioxide from tetraethoxy orthosilane, and obtain the of drug molecules-chelated nanoparticles.

As shown in Step 104 of FIG. 1, after the nanoparticles are formed, the ethanol is used to wash the nanoparticles for several times, to remove the un-reacted chemical substances on the surface of nanoparticles. Now, the core of the present invention is formed.

As shown in Step 105 of FIG. 1, the precursor of reactant such as iron oxide precursor (magnetic precursor, such as FeCl₂ or FeCl₃) is added. Due to the structure-directing effect of Polyvinylpyrrolidone, the iron ion will deposit, by adsorption, on the surface of nanoparticles. A self-assembly process will proceed to form a thin shell. The said thin shell is reduced to form iron oxide (the magnetic-sensitive) shell through a redox reaction.

As shown in Step 106 of FIG. 1, the ethanol is used to remove the un-reacted chemical substances on the surface of nanoparticles to obtain the nano single-crystal shell structure. Now, the shell structure of the present invention is formed. It is the main feature of the present invention.

In addition, in the second embodiment of the present invention, as shown in Step 101 of FIG. 1, firstly, the polymer is added, such as the Polyvinylpyrrolidone (PVP) is dissolved in organic solvent.

As shown in Step 102 of FIG. 1, the drug molecules (the fluorescence molecules can be simulated as the drug molecules) are mixed with the aforesaid organic solution to conduct the hydrolysis for several hours.

As shown in Step 103 of FIG. 1, the nano sphere is obtained from the Polyvinylpyrrolidone after some time, and the drug molecules-chelated nanoparticles are obtained.

As shown in Step 104 of FIG. 1, after the nanoparticles are formed, the ethanol is used to wash the nanoparticles for several times, to remove the un-reacted chemical substances on the surface of nanoparticles. Now, the core of the present invention is formed.

As shown in Step 105 of FIG. 1, the precursor of reactant such as iron oxide precursor (magnetic precursor, such as Fe(acac)₃ or Fe(CO)₅) is added. Due to the structure-directing effect of Polyvinylpyrrolidone, the iron ion will deposit, by adsorption, on the surface of nanoparticles. A self-assembly process will proceed to form a thin shell. The said thin shell is reduced to form iron oxide (the magnetic-sensitive) shell through a redox reaction.

As shown in Step 106 of FIG. 1, the ethanol is used to remove the un-reacted chemical substances on the surface of nanoparticles to get the nano single-crystalline shell structure. Now, the shell structure of the present invention is formed. It is the main feature of the present invention.

In addition, in the third embodiment of the present invention, as shown in Step 101 of FIG. 1, firstly, the polymer is added, such as the Polyvinyl Alcohol (PVA) is dissolved in organic solvent.

As shown in Step 102 of FIG. 1, the drug molecules (the fluorescence molecules can be simulated as the drug molecules) are mixed with the aforesaid organic solution to conduct the chelate reaction for several hours.

As shown in Step 103 of FIG. 1, the nano sphere is obtained from the Polyvinyl Alcohol after some time, and the nanoparticles of chelated drug molecules is obtained.

As shown in Step 104 of FIG. 1, after the nanoparticles are formed, the ethanol is used to wash the nanoparticles for several times, to remove the un-reacted chemical substances on the surface of nanoparticles. Now, the core of the present invention is formed.

As shown in Step 105 of FIG. 1, the precursor of reactant such as iron oxide precursor (magnetic precursor, such as Fe(acac)₃ or Fe(CO)₅) is added. Due to the structure-directing effect of Polyvinylpyrrolidone, the iron ion will deposit, by adsorption, on the surface of nanoparticles. A self-assembly process will proceed to form a thin shell. The said thin shell is reduced to form iron oxide (the magnetic-sensitive) shell through a redox reaction.

As shown in Step 106 of FIG. 1, the ethanol is used to remove the un-reacted chemical substances on the surface of nanoparticles to get the nano single-crystal shell structure. Now, the shell structure of the present invention is formed. It is the main feature of the present invention.

In addition, in the fourth embodiment of the present invention, as shown in Step 101 of FIG. 1, firstly, the polymer is added, such as the Poly (lactide-co-glycolide) (PLGA) is dissolved in organic solvent.

As shown in Step 102 of FIG. 1, the drug molecules (the fluorescence molecules can be simulated as the drug molecules) are mixed with the aforesaid organic solution to conduct the chelate reaction for several hours.

As shown in Step 103 of FIG. 1, the nano sphere is obtained from the Poly (lactide-co-glycolide) after some time, and the drug molecules-chelated nanoparticles are obtained.

As shown in Step 104 of FIG. 1, after the nanoparticles are formed, the ethanol is used to wash the nanoparticles for several times, to remove the un-reacted chemical substances on the surface of nanoparticles. Now, the core of the present invention is formed.

As shown in Step 105 of FIG. 1, the precursor of reactant such as iron oxide precursor (magnetic precursor, such as Fe(acac)₃ or Fe(CO)₅) is added. Due to the structure-directing effect of Polyvinylpyrrolidone, the iron ion will deposit, by adsorption, on the surface of nanoparticles. A self-assembly process will proceed to form a thin shell. The said thin shell is reduced to form iron oxide (the magnetic-sensitive) shell through a redox reaction to form the said nano-carrier with a drug-containing composite core surrounding with a thin magnetic iron oxide shell.

As shown in Step 106 of FIG. 1, the ethanol is used to remove the un-reacted chemical substances on the surface of nanoparticles to get the nano single-crystalline shell structure. Now, the shell structure of the present invention is formed. It is the main feature of the present invention.

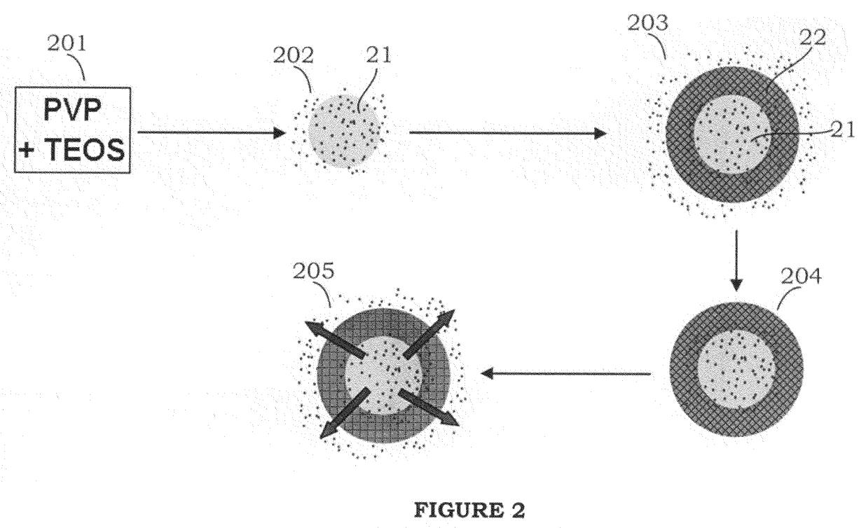

FIG. 2 shows the simulation diagram of the present invention. Label 201 of FIG. 2 shows the result of Step 101 of the present invention, which is the result by dissolving the Polyvinylpyrrolidone and Tetraethoxy orthosilane in the aqueous solution.

Label 202 of FIG. 2 shows the result of Step 102 of the present invention, which is the result by mixing the drug molecules with the aforesaid aqueous solution to conduct the hydrolysis for several hours. The core 21 of Label 202 is composed of the Polyvinylpyrrolidone, silicon dioxide and drug molecules.

Label 203 of FIG. 2 shows the result of Step 103, Step 104 and Step 105 of the present invention. The shell 22 is single-crystalline iron oxide.

Label 204 of FIG. 2 shows the result of Step 106 of the present invention, which is the result by using the ethanol to wash the nanoparticles for several times.

Label 205 of FIG. 2 shows the simulation result of releasing drug by the magnetic control.

The present invention relates to a core-shell nano-carrier having a drug-containing nanocomposite core surrounding with a single-crystal magnetic iron oxide shell, comprising:

The organic material/inorganic material and drug molecules are first reacted to form a drug-containing nanocomposite core structure. Then, the precursor ions of the reactant are grown on the core surface of nanocomposite via polymer targeting by controlling the concentration, time and temperature of reactant to form a nano drug-carrier capsule having the magnetic single-crystal shell.

This process can be reacted at room temperature. This core-shell nano-carrier not only can protect the drug molecules, but also can encapsulate the drug molecules in the core completely, to reach zero-release effect. It has an excellent magnetic sensitivity. The release speed of drug can be controlled from almost zero-release to large amount release by the magnetic field. So it is an excellent drug control and release system.

The present invention uses the organic material/inorganic material and drug molecules to react to form a drug-containing nanocomposite structure. The polymer is used to control the growth of magnetic crystalline.

The core-phase of drug container of the present invention can be composed of the organic materials such as polymers, drugs, inorganic materials such as oxides, glasses, nanotubes, or organic/inorganic composites.

The size of the said nanoparticle core formed by the reaction of the organic/inorganic precursors and drug molecules can have a range of 1 nm to 5000 nm. Except the circular shape, the core can be designed into various geometry.

The drug encapsulated in the drug container formed by the reaction of the organic material/inorganic material and drug molecules can be fluorescence molecules, hydrophilic or hydrophobic drug molecules, biomolecules and functional substances.

In the core-shell drug-carrier of the present invention, a single-crystal magnetic shell can be formed on the nanoparticle to form a drug-containing nanocomposite core surrounding with a single-crystal magnetic iron oxide shell. The magnetic nano-structure can be developed into single crystal, multiple crystalline or non-crystalline or amorphous structures.

In the core-shell drug-carrier of the present invention, a single-crystal magnetic shell can be formed on the nanoparticle to form a drug-containing nanocomposite core surrounding with a single-crystal magnetic iron oxide shell. The thickness of shell can be from 1 nm to 5000 nm. The shape of outer shell can be other shape.

In the core-shell drug-carrier of the present invention, the nanoparticles are formed. Then a single-crystal magnetic (such as iron oxide) shell can be formed on the nanoparticle to form a core (drugs)-shell (magnetic single crystalline) nano-carrier. The substance to form the core-phase can be other material, such as quantum point, metal or polymer.

The making process of the present invention can be reacted at room temperature, but it can be reacted from 0° C. to 300° C. The solvent can be water or organic solvent.

The single-crystal magnetic shell used in the present invention can be magnetic material, such as Fe₂O₃, Fe₃O₄, CoFe₂O₄, MnFe₂O₄, Gd₂O₃ etc., wherein the iron oxide such as Fe₂O₃, Fe₃O₄ is the best, due to simpler process and lower cost and excellent magnetic sensitivity.

The precursor used in the present invention includes but not limits to the following chlorides such as FeCl₂, FeCl₃ and CoCl₂; nitrates such as Fe(NO₃)₂; acetates such as Fe(CH₃COO)₃, Co(CH₃COO)₂ and Mn(CH₃COO)₂ etc.

Therefore, the method for forming a magnetic drug-carrier nanocapsule with a thin magnetic-sensitive shell is described as the followings:

Firstly, forming a drug nanocarrier is carried out, that is an organic and inorganic core with one type of drug molecule, wherein the organic and inorganic core is a nanoparticles core of the drug nanocarrier. Then, a structural-directing molecule is deposited on the drug nanocarrier, wherein the structural-directing molecule is used to induce a precursor of reactant to directly grow up on a surface of the drug nanocarrier; and finally, an in-situ redox reaction is achieved to form the drug-carrier nanocapsule with the thin magnetic-sensitive shell.

In addition, a magnetic drug-carrier nanocapsule with a thin magnetic-sensitive shell will comprise the followings:

A drug nanocarrier which is an organic and inorganic core with one type of drug molecule, wherein the organic and inorganic core being a nanoparticles core of the drug nanocarrier; and

A structural-directing molecule is deposited on the drug nanocarrier, wherein the structural-directing molecule is used to induce a precursor of reactant to directly grow up on a surface of the drug nanocarrier.

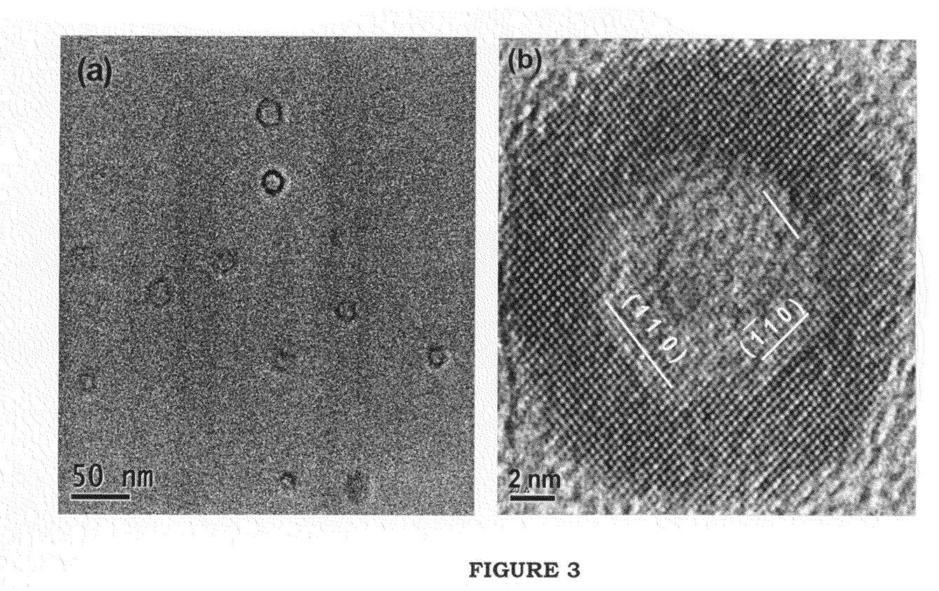

FIGS. 3(a), 3(b) show the Transmission Electron Microscopy image of the core-shell nano-carrier of the present invention. It is shown that the alignment of crystal lattice is very regular, and the thickness is even.

The drug-carrier with magnetic sensitivity is prepared in the present invention. The process technology of nano-material is used to control the carrier structure to get the best feature. The drug carrier of the present invention can encapsulate drug in the core, and use nano technology to encapsulate drug in the single-crystal shell. In addition, the present invention can be finished at room temperature, which will not destroy the activity of drug.

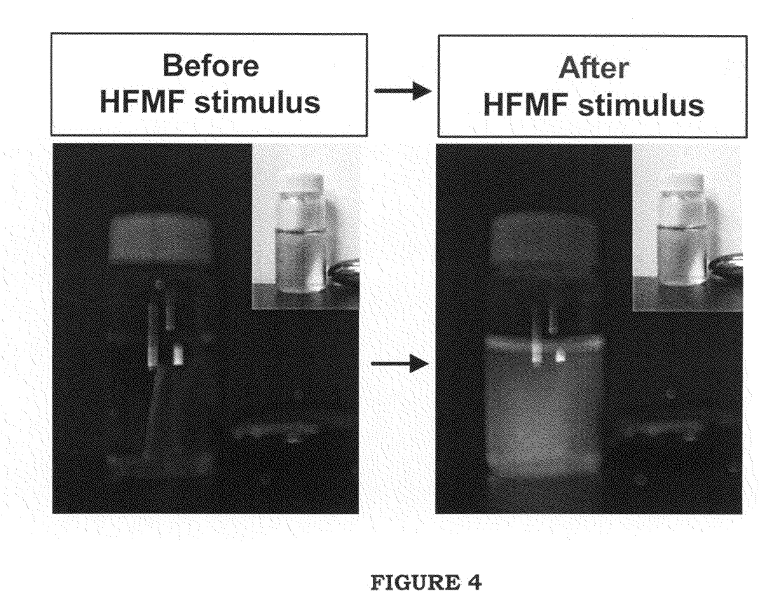

The drug-carrier capsule with the magnetic nano single-crystal shell has an excellent magnetic sensitivity. As shown in FIG. 4, the fluorescence dye is used as a model drug and encapsulated in the core for the test of magnetic sensitivity. The fluorescence test shows that the drug can be encapsulated in the core by the carrier continuously in the absence of magnetic stimuli. When the magnetic field is applied to the drug-carrier, a great amount of fluorescence dye can be released quickly and precisely by the control of magnetic field. This feature is excellent for the long-term drug control.

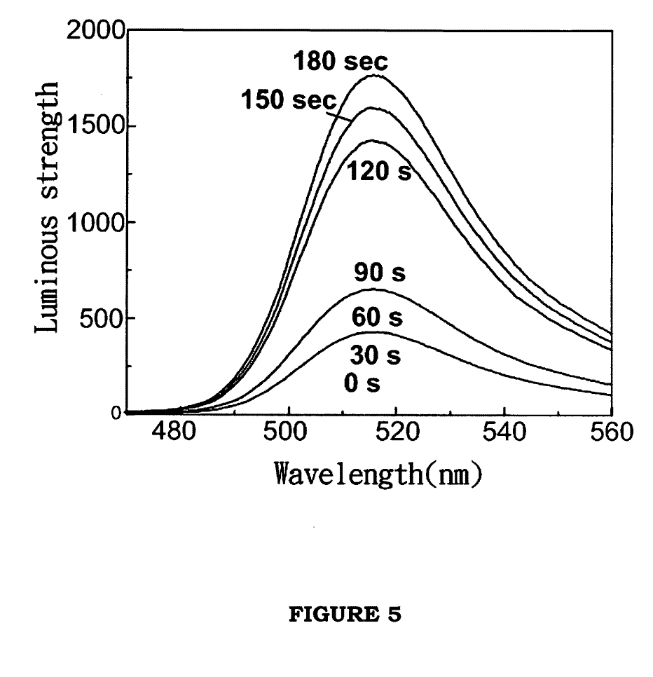

And FIG. 5 further demonstrates that a short-time stimulation of the magnetic field, the magnetic nano single-crystal iron oxide capsule can reach a fast drug-release reaction. It shows the excellent manipulation feature of nano drug-carrier of the present invention. It can be applied to kill the tumor cells or prevent the outbreak chronic disease such as the epilepsy.

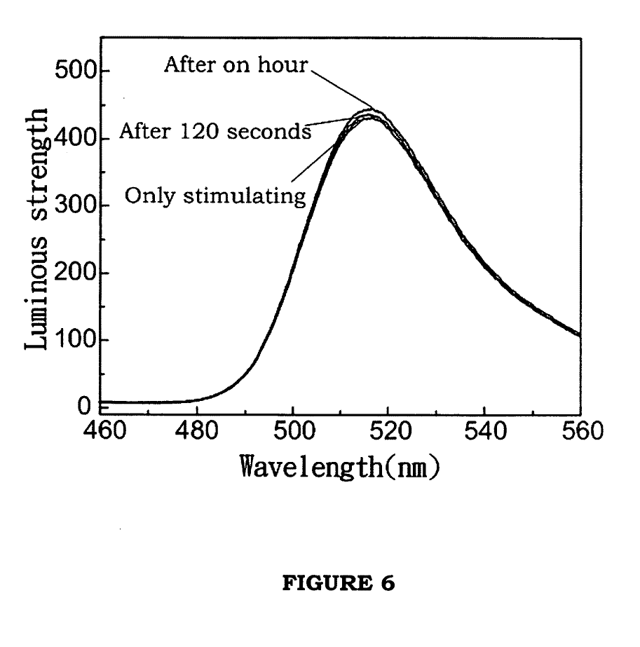

FIG. 6 is the result for the zero-releasing of drug. The magnetic nano single-crystal iron oxide capsule is stimulated by the magnetic field for 60 seconds at first. Later, the magnetic field is moved immediately and the release situation of fluorescence molecules is observed. The result of FIG. 6 shows after it is stimulated by the magnetic field for 60 seconds, the fluorescence molecules signal of solution can reach certain intensity rapidly, which shows part of fluorescence molecules have been released quickly. However, when the magnetic field is removed, it is found that the variation in the luminescent intensity of fluorescence molecules is very small after short time such as 120 seconds or long time such as an hour. The result shows when the magnetic field is removed, the fluorescence molecules can be encapsulated in the magnetic nano single-crystal iron oxide capsule completely without releasing. It means that this carrier is sensitive to the magnetic field. As the switch of the magnetic field can react on the behavior of drug release immediately, the drug release feature is controlled by the magnetic field, which has excellent response effect.

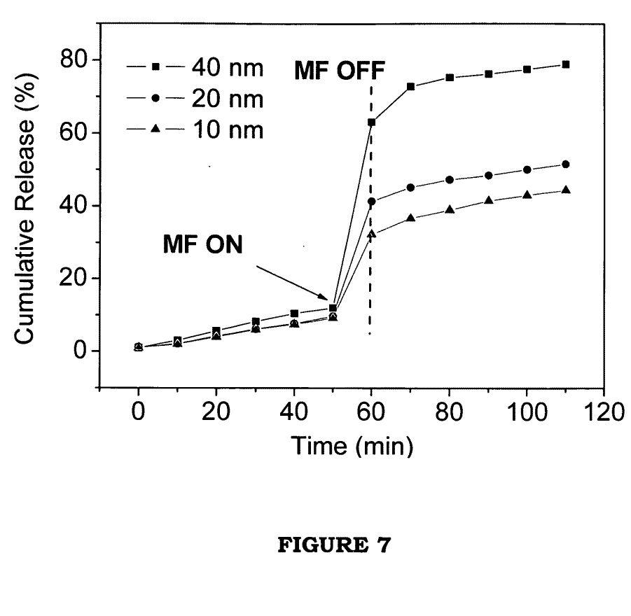

As shown in FIG. 7, the magnetic single-crystal iron oxide capsule with different nanoparticle size can get different drug releasing curve under the same magnetic field. It is known that the drug release feature of single-crystal magnetic iron oxide shell depends on particle size. The single crystalline iron oxide shell with different particle size can respond different magnetic field, so the drug release of drug carrier is different under the same magnetic field.

The drug molecules can be released rapidly under the stimulation of magnetic field. This invention can further integrate with the biological compatible chip to reduce inconvenience of taking drug for patients regularly, and utilize the stimulation signal of living beings to give drug, which can reduce unnecessary drug dosage, and reduce the human injury.

The results show that the amount and mode of drug release can be controlled by the magnetic field and the concentration and size of nanoparticles in the intelligent drug-carrier. The development of integrated drug release system can be widely applied is various diseases, especially the chronic diseases (such as the diabetes) or suddenly occurred disease (heart disease, epilepsy and hypertension). Regardless of giving drugs of the long-term set time, or detect and examine the pathology signal fast, and then the fast reaction reaches the patients for drugs in the body, which can all reach a good result.

It is understood that various other modifications will be apparent and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

What is claimed is:

1. Method for forming a magnetic drug-carrier nanocapsule with a thin magnetic-sensitive shell, comprising:
(a) forming a drug nanocarrier which is an organic and inorganic core with one type of drug molecule, wherein said organic and inorganic core being a nanoparticles core of said drug nanocarrier;
(b) depositing a structural-directing molecule on said drug nanocarrier, wherein said structural-directing molecule being used to induce a precursor of reactant to directly grow up on a surface of said drug nanocarrier; and
(c) inducing an in-situ redox reaction to form said drug-carrier nanocapsule with said thin magnetic-sensitive shell.

2. The method according to claim 1, wherein the nanoparticles core of drug nanocarrier is selected from the group consisting of organic polymer, inorganic material, and drug molecules.

3. The method according to claim 2, wherein said organic polymer comprises polyvinylpyrrolidone (PVP).

4. The method according to claim 2, wherein said inorganic material is oxide selected from the group consisting of silicon dioxide, and titanium dioxide.

5. The method according to claim 2, wherein said drug molecules is selected from the group consisting of fluorescence molecules, hydrophilic, hydrophobic drug molecules, biomolecules, and functional substances.

6. The method according to claim 1, wherein the diameter of nanoparticles core of said drug nanocarrier comprises from about 1 nm to 5000 nm.

7. The method according to claim 1, wherein the shape of nanoparticles core of said drug nanocarrier comprises circular and other arbitrary shape.

8. The method according to claim 1, wherein the material for said magnetic drug-carrier nanocapsule with said thin magnetic-sensitive shell is selected from the group consisting of single crystalline, multiple crystalline, and non-crystalline materials.

9. The method according to claim 1, wherein the shape of outer shell of magnetic drug-carrier nanocapsule with said thin magnetic-sensitive shell comprises the other kind of shape.

10. The method according to claim 1, wherein the substance formed on said magnetic drug-carrier nanocapsule with said thin magnetic-sensitive shell is selected from the group consisting of quantum point, metal and polymer.

11. The method according to claim 1, wherein the reaction temperature of said method for forming said magnetic drug-carrier nanocapsule with said thin magnetic-sensitive shell is under room temperature.

12. The method according to claim 1, wherein said method for forming said magnetic drug-carrier nanocapsule with said thin magnetic-sensitive shell is reacted from about 0° C. to 300° C.

13. The method according to claim 1, wherein the solvent of said method for forming said magnetic drug-carrier nanocapsule with said thin magnetic-sensitive shell is water.

14. The method according to claim 1, wherein the solvent of said method for forming said magnetic drug-carrier nanocapsule with said thin magnetic-sensitive shell is organic solvent.

15. The method according to claim 1, wherein the materials for said magnetic drug-carrier nanocapsule with said thin magnetic-sensitive shell is magnetic materials selected from the group consisting of Fe₂O₃, Fe₃O₄, CoFe₂O₄, MnFe₂O₄, and Gd₂O₃.

16. The method according to claim 1, wherein said precursor of reactant is chlorides selected from the group consisting of FeCl₂, FeCl₃, and CoCl₂.

17. The method according to claim 1, wherein said precursor of reactant comprises Fe(NO₃)₂.

18. The method according to claim 1, wherein said precursor of reactant is acetates selected from the acetate group consisting of Fe(CH₃COO)₂, Fe(CH₃COO)₃, Co(CH₃COO)₂, and Mn(CH₃COO)₂.

19. A magnetic drug-carrier nanocapsule with a thin magnetic-sensitive shell, comprising:
a drug nanocarrier which is an organic and inorganic core with one type of drug molecule, wherein said organic and inorganic core being a nanoparticles core of said drug nanocarrier; and
a structural-directing molecule deposited on said drug nanocarrier, wherein said structural-directing molecule being used to induce a precursor of reactant to directly grow up on a surface of said drug nanocarrier.
